(19) United States
(12) Patent Application Publication
(10) Pub. No.: US 2016/0199602 A1
(43) Pub. Date: Jul. 14, 2016

(54) ADAPTER FOR AN ANESTHESIA MASK OR OTHER MASK

(71) Applicant: Alfredo Fernandez, Miam, FL (US)

(72) Inventor: Alfredo Fernandez, Miam, FL (US)

(21) Appl. No.: 15/076,430

(22) Filed: Mar. 21, 2016

Related U.S. Application Data
(63) Continuation-in-part of application No. 14/188,000, filed on Feb. 24, 2014.
(60) Provisional application No. 61/896,342, filed on Oct. 28, 2013.

(51) Int. Cl. A61M 16/00; A63F 13/26; A63F 13/212; A61M 16/06; A61M 16/01

(52) U.S. Cl. CPC A61M 16/0003; A61M 16/06; A61M 16/01; A63F 13/212; A63F 13/26; A61M 2205/50; A61M 2209/00

(57) ABSTRACT

The present invention relates to an adapter for use with an anesthesia mask or other mask used to administer inhalational gas(es) to a patient. The adapter is structured for providing an interactive video game to a patient before and/or while gas is being administered. The invention further comprises a sensor, and in most embodiments, will be coupled directly or indirectly to a portion of the mask. The sensor or sensor module comprising a microphone and/or other structure for receiving control commands from the patient or user, and a computing device in a communicative relationship with the sensor structured for presentation of interactive media to the user. The sensor module is configured to detect control commands from a patient and relay it as an input signal to the computing device. The computing device is structured to correspondingly alter the interactive media according to the control commands given by a user.

### CLAIM OF PRIORITY

This application is a continuation-in-part of a currently pending U.S. patent application having Ser. No. 14/188,000 and a filing date of Feb. 24, 2014, which made a claim of priority to a U.S. provisional patent application having Ser. No. 61/896,342, filed on Oct. 28, 2013. Each of the above prior-filed applications is hereby incorporated by reference in its entirety.

### BACKGROUND OF THE INVENTION

1. Field of the Invention

This invention is directed to an adapter including a sensor module, as well as accompanying systems and methods, for providing an interactive video game during the administration of anesthesia or other inhalational gases or nebulized medications to a patient.

2. Description of the Related Art

A person who is about to undergo a surgical or other medical procedure often experiences some level of stress and anxiety. This is true for many adults, but is likely to be especially true for a child. For example, being in a hospital, and/or being about to undergo a medical procedure or surgery, is all part of a perioperative experience that can be an extremely stressful and anxiety producing period, particularly for a child. Unfamiliar surroundings and medical personnel combined with uncertainty and the possibility of a parent not being present are all factors that contribute to a young patient's anxiety.

In this situation, it is particularly stressful to prepare a young patient for and to begin the administration of general anesthesia. For example, general anesthesia typically requires transportation of a child from the preoperative area to the operating room, which can be one of the most stressful events in perioperative care regardless of parental presence. To compound the child's anxiety is the placement of an anesthesia face mask over the child's nose and mouth. Most children do not accept placement of the face mask and will resist the anesthesia provider. This can lead to undue stress and heightened anxiety for the child, carrying with it the potential risk for some long-term side effects including post-traumatic stress disorder. In addition, following application of the mask, anesthetics may be delivered to the child to induce an unconscious state through an anesthesia breathing circuit connected to an anesthesia delivery machine that delivers inhalational anesthetics and/or other gases, such as oxygen, in very precise concentrations. Consequently, there is a need to achieve the administration of anesthesia while causing the patient as little stress or anxiety as possible.

Past attempts to address and attenuate a young patent's anxiety have focused on administering either an intravenous, intranasal, or oral dose of a short-acting drug, such as Midazolam. Midazolam is a benzodiazepine, which produces sedation with the purpose of minimizing anxiety and stress. Unfortunately, Midazolam has potential drawbacks such as over-sedation, paradoxical reactions, prolonged anesthesia recovery time, and overall increased healthcare costs.

Success at achieving perioperative mask acceptance by the child without use of sedative drugs has been quite limited to date at best, such as through the use of televisions, scented masks, toys, music and the like. Traditional video games have shown to have somewhat better impact, but carry significant drawbacks for requiring the use of the child's hands for operation of a controller or tablet.

Accordingly, the inventor herein perceives a need for an invention that conquers or at least alleviates the fear often associated with an anesthesia face mask by wielding the enjoyment of playing a video game, while achieving the goal of effectively introducing the mask to the child as a non-threatening object. If any such invention were developed, it would ideally also be capable of distracting the patient from the surroundings by way of a video game, and further, would ideally be structured so as to allow for command control inputs from the patient wearing the mask so as to interact with the video game and cause characters or objects to move within the game, without the use of his/her hands. Ideally, any such invention would also serve to both calm and distract the patient while encouraging the patient to utilize breathing patterns that result in a safer and more effective anesthetization. Moreover, any such invention would ideally also be capable of use in other situations where gases or other nebulized medications are inhaled, such as but not limited to, children being treated for asthma or other medical issues using an inhalational drug delivered by a nebulizer.

### SUMMARY OF THE INVENTION

The present invention addresses these and other needs which exist in the art and in at least one embodiment, is directed toward an adapter that is primarily intended to be used with an anesthesia face mask prior to a medical procedure, such as but not limited, to surgeries on children and other pediatric medical procedures requiring anesthetization of a user or patient through use of an anesthesia face mask. The present invention could also be utilized on adults who are about to undergo a medical procedure requiring anesthetization and also, could be utilized in other situations where gases are to be inhaled, such as but not limited to, a child being treated for asthma using inhalational gases delivered by a nebulizer machine.

As such, in at least one embodiment the adapter of this invention comprises a housing that is sized and configured to be readily and easily connected to an anesthesia mask or another type of mask worn by a patient to inhale one or more gases. The adapter further comprises a sensor module, which may include a microphone, by way of a non-limiting example, that is structured to capture the sounds produced by the user. The sensor module is in communication, whether by wire or wirelessly, with a computing device, which may be a tablet, personal computer, etc., structured for the presentation of interactive media to the user, so that the user is able to interact with and control aspects of the media presented by the computing device. The interactive media will ideally comprise a video game, but can include various other types of visual content.

By way of example only, one type of mask to which the adapter may be connected is an anesthesia face mask that is currently sold under the trademark “Vital Signs,” made by GE Healthcare (a division of GE Technology Infrastructure, itself a division of General Electric) headquartered in Little Chalfont, United Kingdom. With regard to this type of mask, the adapter of the present invention comprises in another embodiment (and as shown in the appended drawings), a housing having a proximal end with a cooperatively structured entry port for facilitating connection of the housing to the aperture of the anesthesia face mask structured for the intake of inhalational anesthetics. In at least one further embodiment, the adapter housing is cooperatively structured to be removable from the mask, the purpose of which will be discussed more fully below.

With regard to the embodiment of the present invention intended for use with an anesthesia face mask, the adapter can include a housing that has a centrally located aperture and can also include a sensor. The sensor may comprise a microphone or a flow meter, which are intended to be non-limiting examples of elements or members that a sensor may comprise in accordance with the present invention. In at least one embodiment, the sensor is disposed at and/or in a distal end of the adapter. A microphone, if present, is structured to capture the sounds produced by the user. A flow meter, if present, is structured to capture air flow produced by the user, such as by inhalation or exhalation. The sensor is in communication, whether by wire or wirelessly, with a computing device, which may be a tablet, personal computer, etc., structured for the presentation of interactive media to the user. The user is able to interact with, i.e., to control aspects of, the media presented by the computing device through control commands. The interactive media will ideally comprise a video game, but can include various other types of visual content. In the case of a video game, the user may control characters or objects on a screen or display of the computing device through various control commands. Control commands may comprise spoken orders, words, sounds, breaths, a series or sequences of breaths, or any combination thereof. In at least one embodiment of the invention, such as one representative early prototype of the present invention, sounds and utterances produced by the user will be the primary source of these control commands.

As suggested above, the housing of the adapter is, in at least one embodiment, cooperatively structured to be removable from the mask, whether an anesthesia face mask or other mask. In the embodiment where the adapter is intended for use with an anesthesia face mask, it may be employed long enough to calm the child, familiarize the child with the mask, and complete other necessary pre-operative procedures before removal of the adapter from the mask. Upon removal of the adapter, the normal anesthesia breathing circuit could, in one embodiment, be introduced and/or known components associated therewith into the anesthesia face mask so that administration of inhalational anesthesia gases can begin.

In at least one additional embodiment of the present invention, the computing device associated with the adapter can be placed into an “anesthesia mode” by the anesthesiologist, nurse anesthetist, other attendant medical personnel, operating room staff, etc. The anesthesia breathing circuit is likely to be attached to the anesthesia face mask during this period of time in order for anesthetization of the patient or user, and the adapter of the present invention may be removed as a preliminary step in doing so. As such, in one contemplated “anesthesia mode,” the computing device continues simulating interaction between the user and the interactive media, as if the computing device were still receiving control commands from the user. It should be understood that in the instance where the adapter of the present invention is not removed from the anesthesia face mask but maintained in place for delivery of the inhalational anesthetics, the user may be losing consciousness and no longer able to effectively issue control commands, and consequently, operation of the computer device in an anesthesia mode would also be desirable. Also, operation of the computing device in an “anesthesia mode” may continue until at least such time as the user has become unconscious, although if desired, the anesthesia mode may continue throughout the patient's surgery or medical procedure and/or even during the patient's recovery from anesthesia.

In at least one additional embodiment, the sensor may be alternatively disposed and connected to another portion of the adapter, such as to a side portion of the housing. In this embodiment, the housing has a distal end structured for connection to the anesthesia breathing circuit, and does not have to be removable from the anesthesia face mask. Consequently, in this embodiment anesthesia is able to flow through the adapter and be administered to the patient unimpeded by the sensor. Also in this embodiment, the user is able to continue interaction with the computing device via the sensor during administration of the inhalational anesthetics to the extent he or she is able to effectively make control commands. Accordingly, while the adapter is capable of removal from between the anesthesia face mask and anesthesia breathing circuit, it may be desirable to retain its presence to keep the child engaged in the interactive media during administration of anesthesia until unconsciousness is achieved.

In addition, the adapter of the present invention may be formed of materials that are relatively inexpensive, so that it can be readily disposed after a single use.

Other embodiments of the invention are directed to a sensor module for use with an anesthesia mask but also other masks, as well as to systems and methods for providing an interactive video game during the administration of anesthesia or other inhalational gases to a patient. Accordingly, the sensor module can be attached to, whether permanently or temporarily, to the mask, and configured to receive command controls from the patient. The command controls are relayed as an input signal to a computing device in communication relations with the sensor module, such as to effect the initiation of various game mechanics in an interactive video game, in response to command controls given by the patient and received at the sensor module.

These and other objects, features and advantages of the present invention will become clearer when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1a is a view of one embodiment for an adapter in accordance with the present invention.

FIG. 1b is a perspective view in partial cutaway of the adapter of FIG. 1a.

FIG. 1c is a view of another embodiment of an adapter in accordance with the present invention.

FIG. 2 is a view of the adapter shown in FIGS. 1a and 1b and shown as about to be placed for use in conjunction with an anesthesia face mask, as indicated by the directional arrows.

FIG. 3 is a view of the adapter of FIG. 1c in assembled form with an anesthesia face mask and an anesthesia breathing circuit.

FIG. 4 is an exploded view of the adapter of FIG. 1c about to be placed for use in conjunction with an anesthesia face mask and an anesthesia breathing circuit, as indicated by the directional arrows, and shown in the assembled form of FIG. 3.

FIG. 5 is a schematic view of an embodiment depicting the adapter of FIG. 1a and a computing device.

FIG. 6 is a schematic view of structural and operational elements of an embodiment of the method of the present invention.

FIG. 7 is a perspective view of an adapter according to the present invention in another embodiment comprising a housing and a sensor module either connected to or coupled to a face mask.

FIG. 8 is a schematic view of the adapter shown in FIG. 7 and a computing device.

FIG. 9 is a schematic view of structural and operational elements of the method of the present invention in another embodiment.

Like reference numerals refer to like parts throughout the several views of the drawings.

### DETAILED DESCRIPTION OF THE PREFERRED EMBODIMENT

As represented in the accompanying figures, the present invention is directed in at least one embodiment to an adapter that is primarily intended for use prior to a medical procedure requiring anesthetization of a user through use of an anesthesia face mask, such as but not limited, to surgeries on children and other pediatric medical procedures, as well as those on adults. It is emphasized, however, that the present invention can also be readily used and/or modified for use in situations where another gas or other gases are inhaled, such as but not limited to, when a child needs to be treated for asthma or another medical issue which is/are delivered by a nebulizer machine.

Additionally, the adapter of the present invention may be manufactured from relatively inexpensive materials so as to be disposable after a single use. Accordingly, the adapter may be intended for single-use and be discarded following one anesthesia or other medical procedure.

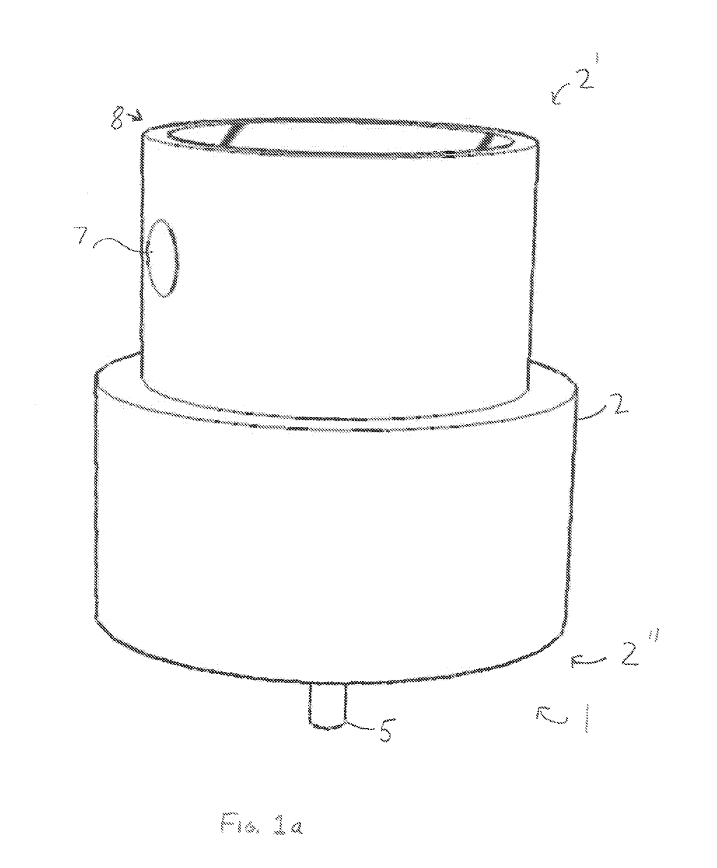

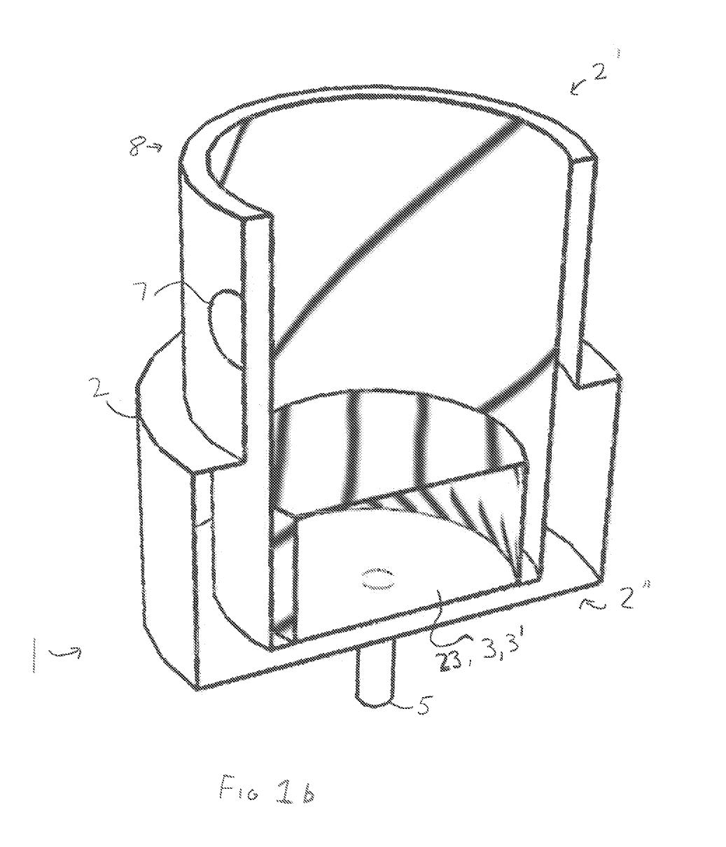

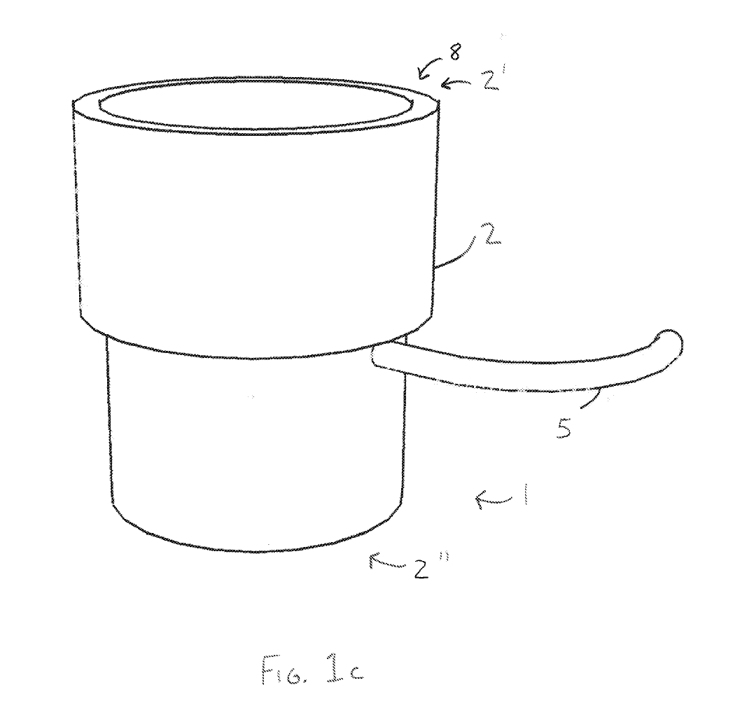

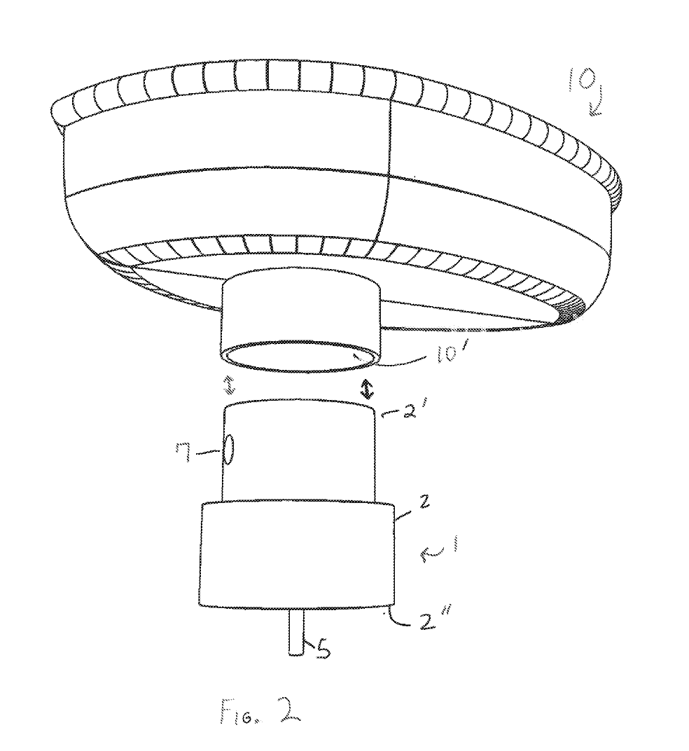

With reference now to FIG. 1a, the adapter 1 of the present invention is shown in a first possible embodiment that is seen to comprise a housing 2, with a proximal end 2′ and a distal end 2″. In this embodiment, the proximal end 2′ of the housing 2 comprises an entry port 8. The entry port 8 is cooperatively structured to facilitate connection of the housing 2 to an anesthesia face mask 10, as shown in FIG. 2. Specifically, the entry port 8 is connected to an orifice 10′ of an anesthesia face mask 10 structured for the intake of a primarily gaseous mixture which may comprise various inhalational anesthetics and gases. As described below, the anesthesia face mask 10 or an equivalent mask may be used without inhalational anesthetics, but the mask would nonetheless include an appropriately structured orifice such as 10′. In at least one embodiment, the entry port 8 is cooperatively structured such that the connection between the entry port 8 and the anesthesia face mask 10 allows for the adapter to be removable. For example, the proximal end 2′ of the adapter 1 can be inserted into the orifice 10′ of mask 10, in the direction of the arrows shown in FIG. 2. The entry port 8 may be curvilinear about its circumference, i.e., having a generally circular cross section or other cross section that is resemblant of a circle or other shape corresponding to the orifice 10′ of the mask 10. Accordingly, the adapter 1 can be readily “plugged into” the anesthesia face mask 10 and removed at the discretion of for example, the anesthesiologist, anesthesiologist assistant, nurse anesthetist, or if the circumstances permit, of operating room personnel or other medical attendants, etc.

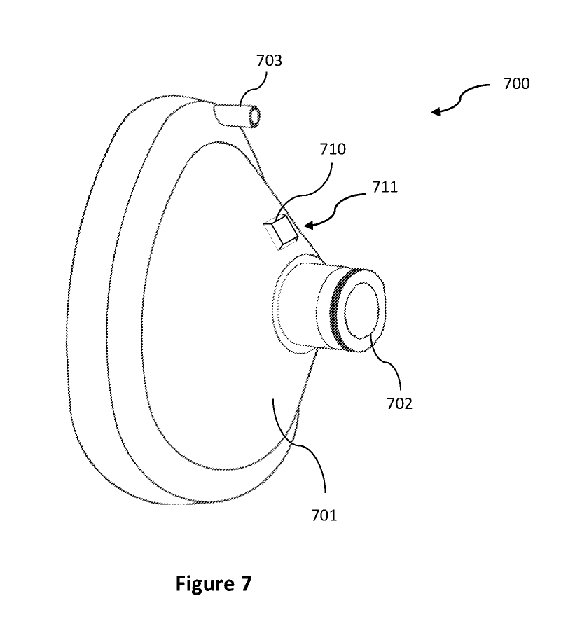

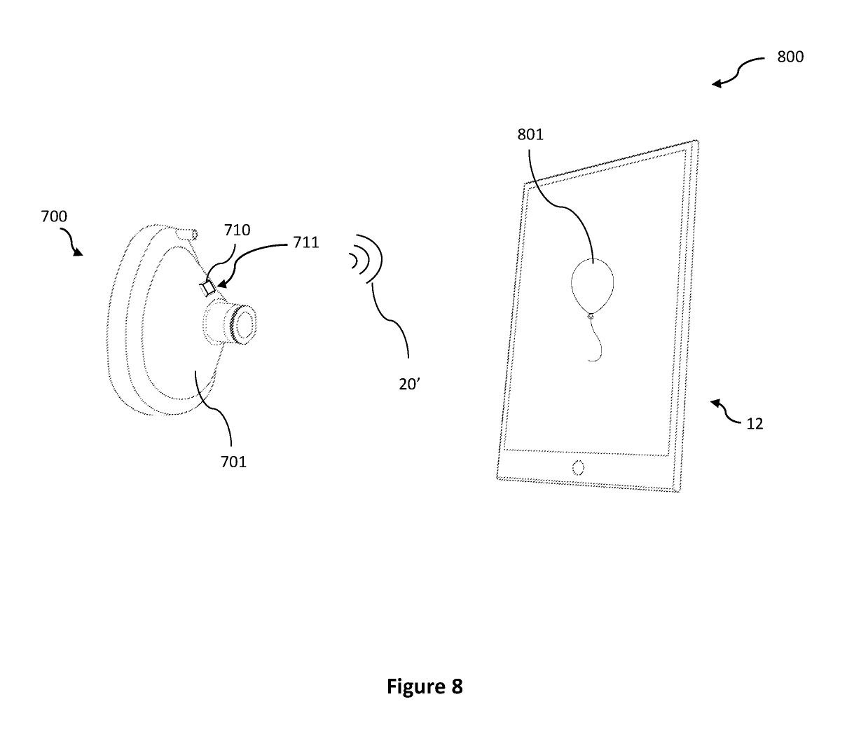

With primary reference now to FIG. 1b, in the illustrated embodiment the housing 2 includes a centrally located aperture. In addition, near the distal end 2′ of the housing, a sensor 23 is disposed at least partially within the housing 2. The sensor 23 may comprise a microphone 3 or a flow meter 3′, the structure and function of which will each be addressed in turn. These are intended to be non-limiting examples of elements or members that a sensor 23 may comprise. It should be appreciated that this may include a sensor module 710 such as described below with reference to FIGS. 7 and 8, including one or more of the structural features described herein, and/or a communications component that may even include a microchip/processor carrying suitably programmed software for communicating with a computing device 12. Other embodiments of sensors 23 may comprise, by way of example, an element for the detection and recordation of eye movement or others members that may become known in the future. It should also be appreciated that, while most embodiments may implement a sensor 23 that comprises either a microphone 3 or a flow meter 3′ but not both, due to space, cost or other constraints, as used herein, it is not intended that the sensor 23 should be limited as necessarily comprising only one of the two elements. Accordingly, at least one embodiment of the present invention may include a sensor that comprises both a microphone 3 and a flow meter 3′.

In the embodiment of the present invention wherein the sensor is a microphone 3, it is oriented such that it captures sounds travelling within the interior of the housing 2, as shown in FIG. 1b. The microphone 3 may be defined as being of a construction that is an acoustic-to-electric transducer or sensor for the conversion of sound to an electrical signal. In addition, the microphone 3 is an appropriately structured member for the detection of audio signals and the conversion thereof to corresponding electronic counterparts.

In another embodiment, the sensor is a flow meter 3′, oriented to detect and measure the flow of air generated by the user. As used herein, the term “air” refers to any primarily gaseous mixture and includes that which a user exhales, inhales, and that which is typically present in the adapter 1. The “flow” of air refers to any currents, perturbations, and other kinetic or dynamic movements within air that may be caused by a difference in pressure. The flow meter 3′ may be a type of mass air flow sensor, volumetric flow sensor, or type of spirometer, such as a pneumotachometer, peak flow meter, windmill-type spirometer, etc., many of which are generally known and commercially available. In any event, the flow meter 3′ is an appropriately structured member to detect the flow of air that the user produces, which the present device will interpret as a control command. Accordingly, this flow of air may be an inhalation, exhalation, breath, etc. that the user produces. The flow meter 3′ converts this flow of air to a corresponding electrical counterpart.

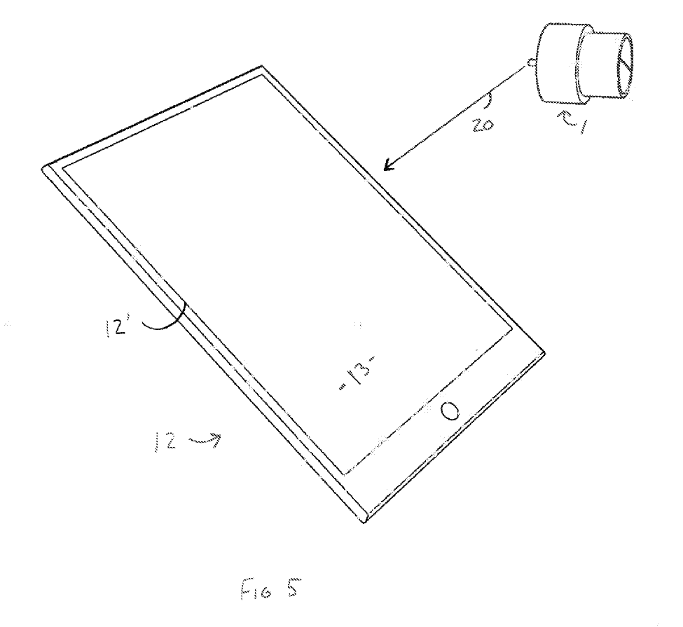

As previously described herein, the sensor 23 may be either wired or wireless. In either case, the sensor 23 is in a communicative relationship with a computing device 12, as shown in FIG. 5, the nature and specifics of which will be discussed in further detail below. As shown in FIG. 1a through FIG. 2, if the sensor 23 is wired, a wire 5 may be appropriately connected to the sensor and extend out of the housing 2 for communicative connection with the computing device 12. In an alternative embodiment, if the sensor 23 utilizes a method of wireless communication including but not limited to WiFi or Bluetooth, the wire 5 may not be present or may instead be replaced by a wireless antenna if the sensor 23 so requires.

In at least one embodiment, such as that of FIG. 1a, the housing 2 may further comprise one or more ventilation apertures 7 passing entirely through a portion of the housing 2. The ventilation aperture 7, which may be located on the side of the housing 2, is structured to allow the passage of air there through when the adapter is in operative use.

In at least one additional embodiment, such as that depicted in FIG. 1c, the sensor 23 may instead be connected to another portion of the adapter, such as on a side portion of the housing 2. In the illustrated embodiment, the housing 2 has a centrally located aperture, but does not necessarily have to be in all embodiments.

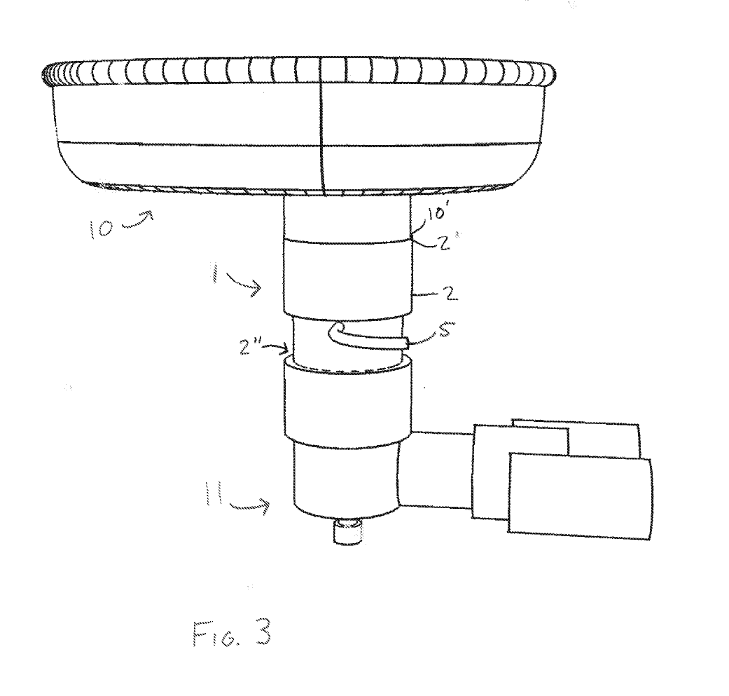

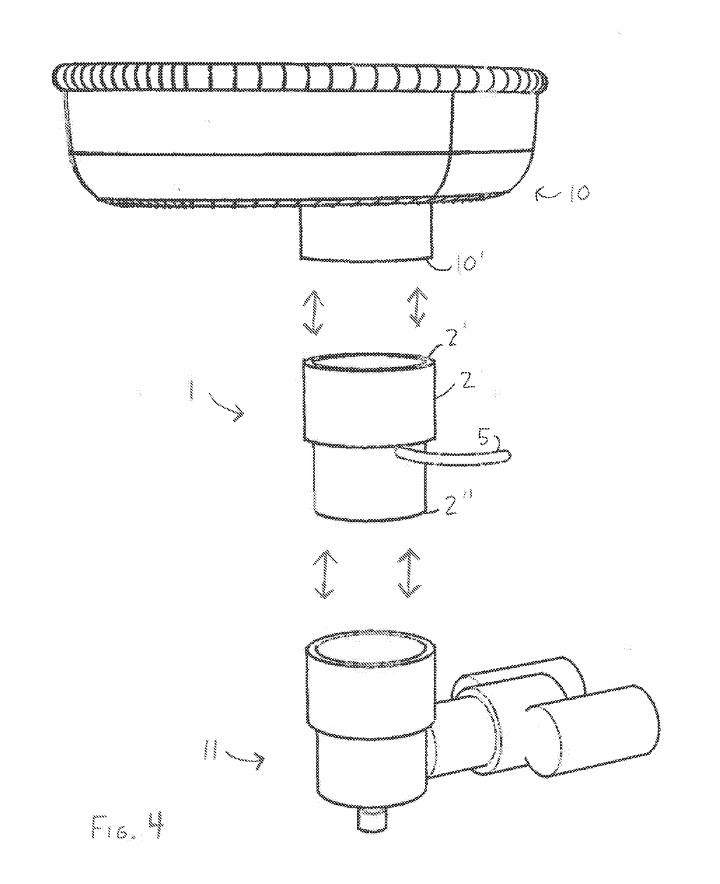

With reference now to FIGS. 3 and 4, the illustrated adapter 1 is shown as having a distal end 2″ of the housing that is cooperatively structured for connection to an anesthesia breathing circuit 11. FIG. 4 illustrates an exploded view of known components for the anesthesia breathing circuit 11 and demonstrates the connection of the adapter 1 in one embodiment directly to the anesthesia breathing circuit 11 and the anesthesia face mask 10 as indicated by the directional arrows. These components are shown in an assembled form in FIG. 3. This connection between the adapter 1 and the components 11 may also be removable. In this embodiment, the adapter 1 allows the unimpeded flow of anesthesia from the anesthesia breathing circuit 11, through the adapter 1, and in turn to the anesthesia face mask 10. As stated above, the sensor 23 (not shown in FIG. 4) may be at least partially disposed within a side of the housing 2. Accordingly, a side portion of the housing 2 may be appropriately apertured to receive the sensor 23, which is appropriately situated and secured therein and which may be wired or wireless.

With primary reference now to FIG. 5, the sensor 23 of the present invention, whether wired or wireless, is in communicative relation with a computing device 12. This communicative relationship is indicated by a directional arrow 20 in FIG. 5 for ease of reference. The computing device 12 may be a tablet, such as an iPad®, cellular telephone, including a smart phone, or may be a personal computer, such as a desktop computer or laptop, a video game system, touchscreen mobile device, wearable electronic device such as a watch or wearable glass or headset, or other appropriate device. The computing device 12 may be also be structured to include installation and the execution and/or running of an Android® or iOS® operating system. If the computing device 12 by its nature lacks an integrated display, an external display, such as a monitor, television, etc., should be connected thereto, and the result shall be considered part of the computing device 12. The computing device 12 comprises hardware sufficient to facilitate the communicative relationship with the sensor 23, a display 12′, and the hardware necessary for the presentation on the display 12′ of interactive media 13. The hardware necessary for the presentation of the interactive media may comprise processor and memory, the latter being in the form of for example, a hard disc drive, solid-state drive, flash drive, or RAM. The computing device 12 may further comprise other hardware, such as a graphics card; inputs for discs such as CDs, DVDs, or other optical discs; input devices for operation of the computing device 12 such as a keyboard or mouse or ports or plugs sufficient for their connection, or other additional hardware components as desired. Should the sensor 23 be wired, the computing device 12 may comprise the appropriate port necessary for connection of the sensor 23 thereto.

Still referring to FIG. 5, the computing device 12 is structured for the presentation of interactive media 13 to the user. Interactive media 13 is intended to mean any media structured to be responsive to user input. Accordingly, the interactive media 13 of at least one embodiment may comprise software installed and structured to operate on the computing device 12. In at least one embodiment, the interactive media 13 may comprise video games. Other examples of interactive media 13 may be puzzles or mazes the user must solve. The user input is provided in the form of control commands that are detected by the sensor 23 during operative placement of the adapter 1 in the anesthesia face mask 10. These control commands, which the user produces, may be voice commands such as words or phrases, variances of tone, pitch or volume of the user's voice, the patient's breathing in and/or exhalations or more forceful “blowing” of air out. Control commands in at least one embodiment may comprise at least some commands that are “utterances,” “babbling,” or other types of non-linguistic sounds unassociated with language. The sensor 23 converts these control commands from their state as audio signals, if the sensor 23 comprises a microphone 3, or as air flow, if the sensor 23 comprises a flow meter 3′, into a corresponding electrical signal that is transmitted to the computing device 12, either through the aforementioned wired or wireless communicative relationship between the sensor 23 and the computing device 12. The interactive media 13, utilizing the hardware of the computing device 12, processes the electronic signals and interprets them as user-generated instructions, or control commands. Accordingly, these control commands facilitate the user's ability to alter the interactive media 13. Control commands may be in the form of voice commands, such as spoken instructions, but do not have to be, as noted previously herein. In the case of a video game, this could mean the user has the ability to move a character or an object about the screen by issuing simple sounds or modified sounds, such as by variance of pitch or by making louder sounds or faster sounds. It could also mean verbal commands. In the case of other media, the verbal commands may comprise specific commands such as saying the word “play” or “start” or other context-appropriate commands. As can be appreciated, the set of verbal commands is structured to correspond to the interactive media 13 being presented.

Additionally or alternatively, control commands may comprise the user blowing into the anesthesia face mask 10 and adapter 1. This blowing would be detected by a flow meter 3′, as noted above. Accordingly, interpretation of the control commands generated by blowing may be done according to aspects of the duration of the exhalation. Additionally or alternatively, the control command issued by such blowing may incorporate aspects directed to multiple breaths within a predetermined portion of time, e.g., the interactive media interprets a particular sequence of breaths as collectively comprising a single control command. Incorporating breaths as control commands may have the additional effect of encouraging a user to adopt desired breathing behavior while using the anesthesia face mask 10, such that subsequent delivery of inhalation anesthetics can be properly accomplished. Training a patient through the use of control commands comprising breathing may facilitate a user's familiarity with the breathing process to ensure that, following connection of an anesthesia breathing circuit 11, the inhalational anesthetics are properly inhaled. Furthermore, in at least one embodiment, the control commands may be structured to facilitate training of the user to help prevent hyperventilation, such as by causing a character in a video game to move improperly, if the user exhales, blows, or inhales too much air or with too much force.

In at least one embodiment, the interactive media 13 may also comprise an “anesthesia mode.” However, anesthesia mode is not a required element for all embodiments of the interactive media 13. Anesthesia mode simulates user input in the absence thereof so that the user believes control commands are still being received and/or recognized by the interactive media 13. This may be desirous so that the adapter 1 can be removed from the anesthesia face mask 10, but the user's attention can remain focused on the interactive media 13 such that the user is distracted during additional pre-operative procedures. Accordingly, the user may continue issuing control commands, believing those commands are still controlling the interactive media 13. The interactive media 13 will, however, in this mode be at least partially self-controlling, simulating user input to distract the user for e.g., at least as long as it takes until the user is rendered unconscious after administration of anesthesia. In at least one embodiment, the simulated user input may be based in part upon the interactive media 13 having learned patterns representative of the user's style of interaction with the interactive media 13, such as the user's play style if the interactive media 13 comprises a video game. Accordingly, the user will observe that the interactive media 13 more closely simulates the user's previous inputs, and the user is less likely to be aware of the at least partial simulation on the part of the interactive media 13. The anesthesia mode may be activated upon removal of the adapter 1 from the anesthesia face mask 10 for the connection of the anesthesia face mask 10 to the anesthesia breathing circuit 11. This “anesthesia mode” may happen automatically if the device 12 detects that the adapter 1 has been removed from the mask, such as by the interactive media 13 detecting the sudden absence of control commands from a user or after not receiving control commands from a user for a predetermined duration of time. Alternatively, operative or medical personnel may activate the anesthesia mode directly, such as by issuing a command directly to the interactive media 13. This could be accomplished e.g., by use of the computing device 12, such as through a keystroke, an input on a touchscreen, a switch on the computing device 12, or any suitable method. Further, activation of anesthesia mode may be done in the moments prior to or after removal of the adapter 1 from the anesthesia face mask 10 or if the adapter 1 is not going to be removed from the mask 10, activation may be accomplished either prior to or during anesthetization of the user.

In at least one embodiment, such as that of FIG. 5 in which the interactive media 13 comprises a video game, the video game may in turn comprise user selectable “power-ups” awarded for a user's performance within the video game. One example of a power-up may be invincibility, such as when a user-controlled character in the video game is impervious to elements, such as non-user-controlled “enemies” or environmental enemies such as on-screen hazards, within the video game that would ordinarily “damage” the user-controlled character. Another example may be a “shield” that allows the user-controlled character to withstand a certain amount of damage, or to cause damage to enemies. These power-ups may be earned by the user, such as by the completion of certain goals or tasks, such as the collection of on-screen items represented by tokens, coins, etc. The power ups may also persist in their effect upon the user-controlled character for a durational limit, such as a number of seconds.

In at least one alternate embodiment, when the aforementioned “anesthesia mode” is engaged, the user-controlled-character may be granted a power-up for the duration of the adapter's status in anesthesia mode. It should be noted that, as used herein, the term “user-controlled character” shall be understood to refer to the character of the interactive media 13 the user can control, including at such times that the character is controlled wholly by the user, or is partially or wholly controlled by the interactive media 13, as described herein. A power-up such as invincibility may be granted to the user-controlled character during anesthesia mode so that the user's character is not damaged while under simulated control, and the user is able to resume control of an intact character upon reawakening. It may also be desired that the user-controlled-character, during anesthesia mode, be able to continue collecting coins, experience points, or other “rewards” that the user can be presented with upon resuming control following the medical procedure.

In at least one alternate embodiment, engagement of “anesthesia mode” permits the user to retain at least partial control of the character until unconsciousness is achieved. Accordingly, the interactive media 13 may gain an appropriately proportionate amount of partial control and “assist” the user in playing the game through the partial control of the character. Upon the user's reaching of an unconsciousness state, the interactive media 13 may assume full control of the character. In addition, anesthesia mode may be implemented to provide the partially user-controlled character with invincibility and/or other power-ups during anesthesia mode, as well as to present the user with “rewards,” such as but not limited to those described above, upon the user's return to consciousness and resumption of control of the character and playing of the game following the medical procedure.

In at least one embodiment, engagement of the interactive media 13 into “anesthesia mode” may be heralded by a “notification” presented to inform the user that the device has been put in anesthesia mode, and administration of inhalational anesthetics has begun or is imminent. As one property of many inhalational anesthetics is an odor, the notification may serve to warn the user before the user is confronted with this odor. The notification may include at least one audial indicator, such as a noise, tones, music, words, or combinations of words. Alternatively or additionally, the notification may include at least one visual indicator, such as text, graphics, pictures, or lights. Alternatively or additionally, the notification may include at least one physical indicator, such as vibration. One possible example of an embodiment of the notification is a graphical representation of a cloud of fog accompanied by the text and/or sentence, read aloud by the interactive media 13, “Here comes the fog!” This example may also include a sound effect, such as a change in the game's music or a fog horn. In such an example, the fog may bear a correlative relationship to an inhalational anesthetic, which may possess an odor that user is confronted with as the graphic of a fog cloud is presented.

In another embodiment, the interactive media 13 of FIG. 5 may comprise an educational story presented to the user before the conduction of the medical procedure. After the presentation of an educational story, the user may be presented with a plurality of video games or stories from which to select. The interactive media 13 then presents the correspondingly selected video game or story to the user. The user may have the option to exit the current selection to reselect a video game or story. The selected video game or story may be presented until placement of the device into anesthesia mode or the user's unconsciousness. The medical procedure would then be conducted during the user's unconsciousness. Upon the user's return to consciousness or the device's exiting anesthesia mode, if the latter is applicable, the user may be presented with a “follow-up” educational story.

In addition, the adapter 1 may also be used in an environment where the patient is being prepared for the delivery of non-inhalational anesthetics, such as those administered intravenously. It is often the practice during the administration of non-inhalational anesthetics for a patient to wear a mask for the delivery of, for example, oxygen during the medical procedure, though no anesthetics are delivered through the mask. Nonetheless, a patient may still experience all the same anxieties associated with use of a mask and preparation for the administration of inhalational anesthetics as previously described. Therefore, use of the adapter 1 with the mask, in this configuration where anesthetics are non-inhalational and delivered in a manner other than through the mask, may still be desirable to distract and calm the user. Accordingly, it is not intended that use of the adapter 1 as described herein should require that the medical procedure involve use of inhalational anesthetics, or that the adapter 1 be so limited. Further, the adapter 1 described herein is not limited to interconnection solely to an anesthesia face mask, as the mask placed over a patient's face during delivery of inhalation of other substances absent anesthesia is nonetheless a mask of the type with which the adapter 1 may be intended to be connected.

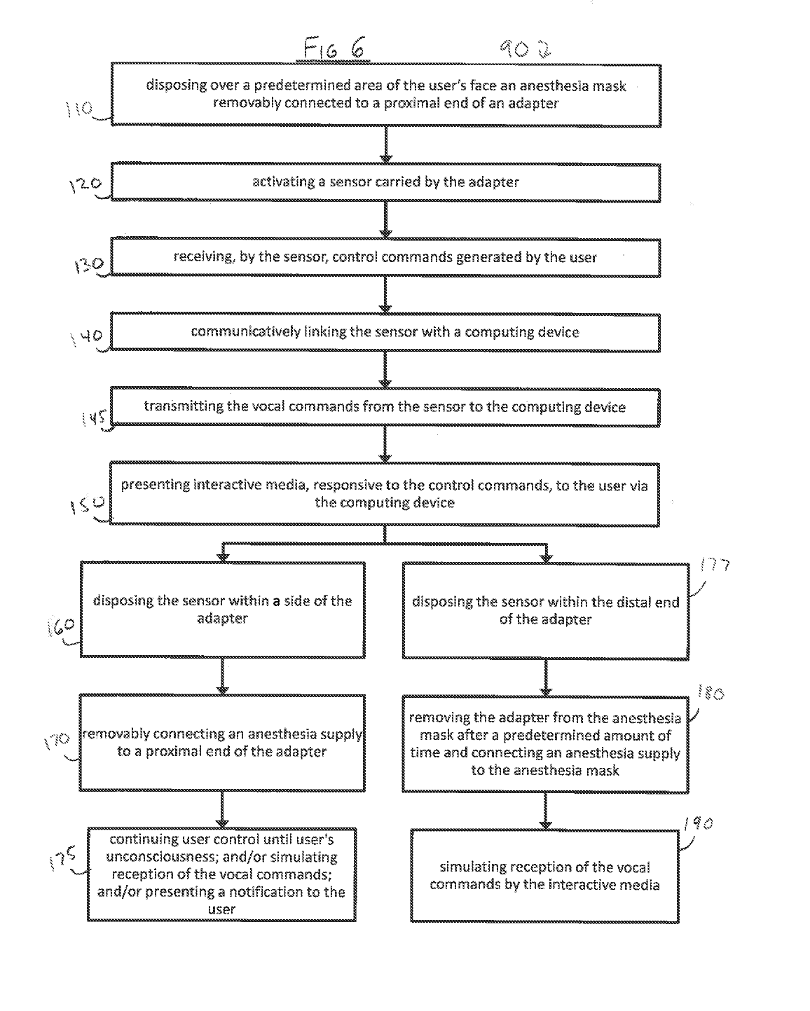

FIG. 6 represents a diagram of an embodiment of a method 90 for preparing a pediatric user for anesthetization. The method 90 comprises disposing over a predetermined area of a user's face an anesthesia face mask, having an adapter removably connected thereto, such as at a proximal end of an adapter, as at 110. A sensor is carried by the adapter, as at 120. Control commands, which are generated by the user, are in turn received by the sensor, as at 130. The sensor is also communicatively linked to a computing device, as at 140. Accordingly, the control commands are transmitted from the sensor to the computing device, as at 145. As previously discussed, this may be achieved through a wireless connection, such as Bluetooth or Wifi, or a wired microphone plugged directly into the computing device. Interactive media, which are displayed on the computing device, are structured to respond to the control commands, as at 150. The interactive media may comprise audiovisual components, video games, etc. and are discussed in more detail above.

In at least one embodiment, the sensor is disposed on another area of the adapter, such as on, near, at or within a side of the adapter, and an anesthesia supply is removably connected to a proximal end of the adapter, as at 170. This facilitates the flow of anesthesia through the adapter. Consequently, the user may continue to issue control commands to the interactive media, as discussed above, during administration of anesthesia.

Additionally, and as indicated at 175, the interactive media may at least partially simulate reception of control commands. In at least one embodiment, the user retains full control until unconsciousness, at which point the interactive media begins simulation. Additionally or alternatively, the interactive media may present a notification to the user of the commencement of anesthesia mode in accordance with the foregoing description. If the interactive media comprises a user-controlled character, the character may be granted a power-up, as described previously herein.

Furthermore, in at least one embodiment, the method 90 comprises disposing the sensor within the distal end of the adapter, as at 177. In addition, an embodiment of the method 90 may comprise removal of the adapter from the anesthesia face mask after a predetermined amount of time to allow the connection of an anesthesia supply to the anesthesia face mask, as at 180. The predetermined amount of time may be at the discretion of the attending medical personnel, and is likely determined according to an amount of time necessary to allow the user to become comfortable with the anesthesia face mask. The method 90 may also comprise the simulation of control commands by the interactive media following removal of the adapter via the “anesthesia mode” previously discussed, as at 190.

Accordingly, as can be appreciated by the foregoing description, the adapter and method described herein can be implemented without requiring participation of a patient's hands. Therefore, due to its potentially hands-free implementation, applications of the adapter 1 may include wound dressing changes, suturing procedures and even certain other surgical procedures done on the hands or other extremities, as well as those performed on patients who have lost the use of one or both hands.

Referring now to FIGS. 7 and 8, the present invention may comprise one or more additional embodiments, and as illustrated, may be directed to a sensor module for connection, attachment, coupling or affixation, whether temporary or permanent, to an anesthesia mask, or another type of mask, as generally illustrated in FIG. 7. The goal of such an embodiment is also to provide an interactive video game during the administration of anesthesia, other inhalational gases or nebulized medications to a patient.

Still referring to FIG. 7, the mask may comprise a mask body 701 and the adapter may comprise an adapter housing 711 which includes a sensor module 710, which are connected to, coupled to or affixed to a portion of the mask body 701. The mask body 701 is shaped to cover at least a portion of a patient's oronasal region. The mask body 701 may comprise at least one air communication port 702 adapted to communicate gas exchange with an external anesthesia gas source. The air communication port 702 may be adapted for reception of an anesthesia gas, and/or for the exhalation of a patient's breath. The mask illustrated in FIG. 7 can also include additional structure 703, such as a nipple that allows for the inflation of soft portions of the mask. Of course, it should be understood that in other embodiments, various anesthesia masks may have only one port or may have more ports, and additional components for recycling the anesthesia gas.

The adapter housing 711 may be connected, coupled or affixed to the mask body 701 such as at an external area thereof, as shown. The adapter housing 711 may comprise a sensor module 710 disposed therein, or alternatively also coupled to a portion of the mask body by temporary or permanent affixation methods as appropriate and known to those skilled in the art. For example, the connection or coupling may be formed by use of an adhesive including but not limited to structural or semi-structural adhesives (e.g., epoxies, cyanoacrylates, urethanes and/or acrylic adhesives); pressure sensitive adhesives via the use of modulus elastomers (e.g., double sided tape); an adhesives paste such as a ballistics gel suitable for sound propagation of a patient's breathing sound or spoken commands; as well as other adhesives known to those skilled in the art. The type of adhesive may be interchangeable based on the type of sensor module 710 being used. Of course, the sensor module 710 and/or adapter housing 711 may also be coupled by other means in addition to the adhesive methods described above, such as by velcro, by suction, or by a physical adapter cooperatively adapted to the anesthesia mask's physical profile or in connecting and communicative relations to its air communication port(s) such as described in the earlier embodiments.

Accordingly, the sensor module 710 is configured to detect control commands from a patient and relay it as an input signal to a computing device running an interactive video game. The sensor module 710 may comprise, without limitation, a pressure sensor (piezoresistive, piezoelectric, capacitive, electromagnetic, optical, potentiometric, resonant, thermal, ionizing, etc.); a temperature sensor (infrared, via use of a thermistor, thermocouple, resistance thermometer, etc.); a sound sensor or microphone; a flow meter; a humidity or moisture sensor; a gas sensor (semiconductor, fuel cell, etc.); and other appropriate sensors known to those skilled in the art. The type of sensor selected may depend on the complexity of control commands that are required to be captured as an input signal, which may in part depend on the complexity of the video game. For example, in a simple video game that merely receives the input signal as binary, i.e., “on” or “off”, with “on” equating to the capture of a patient's breathing out, and “off” with the absence of the capture of such an interpreted “control command” or action, the sensor then required would be a simple one requiring the capture of a single state. However, in more complex video games requiring the capture of different types of control commands or actions by the patient, i.e. such as breathing, the level of breathing, the length of breathing, the intensity of each breath, the incorporation of different voice commands, or various combinations of the above, then more sensitive, more complex, or a combination of the above or other sensors, may be implemented within the sensor module in order to fully capture a wide range of control commands from a patient or user.

At least one embodiment of the present invention is directed to a system 800 for providing an interactive video game during the administration of anesthesia to a patient, as generally illustrated in FIG. 8, or other inhalational gases or nebulized medications. Accordingly, the system 800 comprises an anesthesia mask 700 and adaptor housing 711 including a sensor module 710, such as those described above in communicating relations with a computing device 12.

The adaptor housing 711 including the sensor module 710 may include a communications component capable of wired or wireless communication, i.e., the transmission of instructions or an input signal 20′ to the computing device 12. For example, the sensor module 710 may incorporate therein a wireless communication module such as to facilitate wireless communication via near field communication (RFID, ISO/IEC, GSMA, ETSI/SCP, etc.); Bluetooth; WiFi (802.11 standards including 802.11 a/b/g/n/ac, etc.). Of course, the sensor module 710 may also utilize a wired communications module for communicating with the computing device via Ethernet, coaxial, optical, or other cables or wires capable of transmitting a signal.

The computing device 12, as described above in FIG. 5, may comprise appropriate hardware and software for communicating and receiving an input signal from the sensor module 710 and/or other components disposed within the adapter housing 711. For example, the computing device 12 may comprise a central processing unit (CPU), which may be a single core or multi core processor, memory (random-access memory, read-only memory, and/or flash memory) or primary memory for high-speed storage of executing programs, electronic storage (e.g., hard disk) or secondary memory for storing data, communications interface (e.g., network adapter) for communicating with other devices, such as the sensor module 710 or other computers over a network, and/or peripheral device(s), displays, speakers in communication with the CPU that enable input/output.

The computing device 12 comprises at least one interactive video game or other interactive media (i.e. any audio and/or visual elements that respond to control commands, input signals, or other input received directly or indirectly from a user or patient). The interactive video game is a computer program which includes at least a group of interpretable and/or executable computer code that upon execution, performs the functionality coded therein. Accordingly, the interactive video game may be coded or written in any programmable or interpretable language known to a person reasonably skilled in the art, including but not limited to C, C++, C#, Ruby, Java, Dart, Rust, Swift, PHP, Perl, HTML, XHTML, and other equivalent languages and past, present and future variations. The interactive video game may also be created through use of commercial game makers (applications having various libraries of game mechanics and/or game content) for various iOS and/or Android embodiments, such as GameSalad®, Stencyl, PLAYIR, GameMaker, GameBuilderstudio, Clickteam Fusion, and others, which contain graphic user interfaces for creating video games and various game mechanics therein. However, rather than receiving input signals from traditional input methods, such as touch screen, mouse, keyboard, joystick, etc., the interactive video game will be configured to interface and receive an input signal directly from the sensor module 710 attached to the anesthesia mask 700, therefore making game mechanics respond directly to a user's breathing, voice, and/or other appropriate control commands while wearing an anesthesia mask.

In one illustrative and non-limiting example, the interactive computer program may comprise a game object 801 or in this case, a balloon, that may float higher in relation to a moving background, when an input signal 20′ is received by the computing device 12 from the sensor module 710. The input signal 20′ may correspond to an exhalation of breath by the patient. That is, when the sensor module 710 detects a patient's breath, it transmits an input signal 20′ to the computing device 12 that is executing the interactive video game comprising the interactive balloon. In response to receiving the input signal 20′, the interactive video game will respond by providing visual feedback of the balloon, e.g., with the balloon floating higher and higher, as additional input signals (breaths in this example) are received. Of course, audial feedback may additionally be provided related to the balloon floating higher, such as the playback of a preselected sound file. In some embodiments, physical feedback may also be implemented, i.e. through built-in vibration on the computing device 12.

In one embodiment, more complex input signal 20′ reception and response may be implemented, for example, with a sensor module 710 capable of measuring the intensity of breath or length of breathing, the interactive balloon may be programmed to float higher in response to strong breathing and/or longer breathing. The interactive video game may provide simple instructions or feedback for notify the patient of this, such as to provide an incentive for the patient to blow into the mask more intensely and/or for a longer period, and as a result take deeper breaths (and therefore inhale the anesthesia gas more effectively).

The notification feature may also be implemented to provide the patient with an indicator (visual, audial, physical) on or about commencement of the flow of anesthesia. For example, the interactive video may be programmed to “start” upon the initiation of anesthesia, such as the engagement of an “anesthesia mode” described in the above embodiments. Simple instructions of the video game may be provided to the patient prior to beginning the anesthesia procedure, via the computing device 12. In one embodiment, the interactive video game will be configured or programmed to simulate the receiving of the input signal following the engagement of the anesthesia mode, even when the sensor module 710 is no longer detecting control commands from the patient. In other words, the interactive video game may be programmed to make the patient think that the game is continuing, even as he or she is drifting to sleep. For example, and in the interactive balloon example provided above, this implementation may be created by initiating playback of a animation of the balloon flying above clouds and into space and beyond, when the computing device 12 and/or sensor module 710′ has not detected any input from the user for x seconds, such as a time interval selected from between 1 to 20 seconds.

In more complex video games, different playback animations may be initiated based on the previously received control command(s) from the patient. For instance, if the patient was blowing deeply as instructed, a final animation may be shown of the balloon floating into outer space. However, if the patient was not blowing deeply or was providing a different control commands, such as a series of shallow fast breaths, a final animation may be shown of the balloon floating in a cloud layer.

Of course, any other video game and/or video game character, mechanics, and various complexity, may be implemented with this intended usage of incentivizing a patient to breath and/or breath a certain way, as described in earlier embodiments in this document.

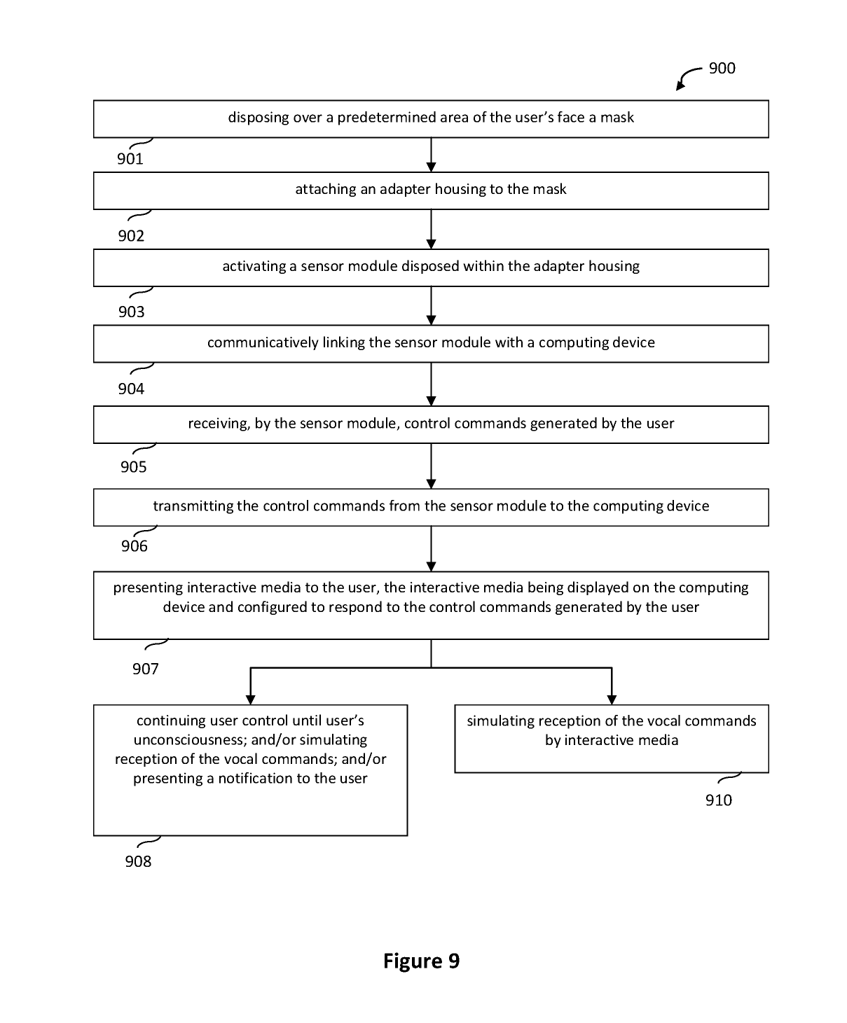

FIG. 9 represents a diagram of an embodiment of a method 900 for either preparing a user to receive anesthesia or another inhalational gas or during the administration of an inhalant or inhalational gas (or both), which may share one or more features of the method 90 recited above in accordance with FIG. 4 in at least one embodiment.

The method 900 comprises disposing over a predetermined area of the user's face a mask, as in 901. The mask may comprise an anesthesia mask or other mask as described within this document. An adapter, such as adapter housing 711 above, is attached to the mask, as in 902. A sensor module, such as sensor module 710 above, disposed within the adapter housing is then activated, as in 903. A communication link is established between the sensor module and a computing device, as in 904. This communication link may comprise wired or wireless communication as described above. The sensor module receives control commands generated by the user, as in 905. The control commands are transmitted from the sensor module and/or adapter housing and/or communications components therein to the computing device, as in 906. Interactive media is presented to the user, as in 907, the interactive media being displayed on the computing device and configured to respond to the control commands generated by the user. In one embodiment, user control may continue until the user's unconsciousness; and/or simulating reception of the vocal commands; and/or presenting a notification to the user, as in 908 and described in additional detail above. In one embodiment, reception of the vocal commands by the interactive media may be simulated, as in 901 and described in additional detail above. Since many modifications, variations and changes in detail can be made to the described preferred embodiment of the invention, it is intended that all matters in the foregoing description and shown in the accompanying drawings be interpreted as illustrative and not in a limiting sense. As just one example, the inventive adapter disclosed herein could also be constructed so as to be employed for re-use, following appropriate and well established disinfectant procedures. As another example, the adapter disclosed herein could also be used by itself, without the anesthesia face mask in certain circumstances, for purposes of hands-free communication with the computing device and interactive media. Thus, the scope of the invention should be determined by the appended claims and their legal equivalents.

Now that the invention has been described,

What is claimed is:

1. An adapter for use with a mask to provide an interactive video game during the administration of an inhalant to a patient, said adapter comprising:
an adapter housing structured and configured to be connected to a mask;
a sensor module disposed within said adapter housing,
said sensor module configured to detect control commands from a patient and relay the control commands as an input signal to a computing device.

2. The adapter of claim 1 wherein said adapter housing including said sensor module is removably coupled to an external surface of the mask.

3. The adapter of claim 1 wherein said adapter housing including said sensor module is affixed to an external surface of the mask.

4. The adapter of claim 1 wherein said sensor module is affixed to an external surface the mask via an adhesive.

5. The adapter of claim 1 wherein said sensor module comprises a pressure sensor.

6. The adapter of claim 1 wherein said sensor module comprises a temperature sensor.

7. The adapter of claim 1 wherein said sensor module comprises a microphone.

8. The adapter of claim 1 wherein said sensor module comprises a flow meter.

9. A system for providing an interactive video game during the administration of an inhalant to a patient, said system comprising:
a mask body shaped to cover at least a portion of a patient's oronasal region while defining an enclosed chamber in air flow communication therewith,
said mask body comprising at least one air communication port adapted to communicate gas exchange with an external inhalational gas source,
a sensor module coupled to a portion of said mask body,
said sensor module configured to detect control commands from a patient and relay it as an input signal to a computing device;
said computing device configured to receive the input signal within an interactive video game and effect an associated action.

10. The system of claim 9 wherein said computing device is a tablet.

11. The system of claim 9 wherein said computing device is a mobile phone.

12. The system of claim 9 wherein said computing device is a wearable electronic device.

13. The system of claim 9 wherein said interactive video game is configured to provide feedback to the patient in accordance to the received input signal.

14. The system of claim 13 wherein the feedback may comprise at least one feedback selected from the group consisting of: an audial feedback, a visual feedback, a physical feedback.

15. The system of claim 14 wherein said interactive video game is configured to notify the patient on or about commencement of the flow of the inhalational gas.

16. The system of claim 15 wherein the notification may comprise at least one indicator selected from the group consisting of: an audial indicator, a visual indicator, a physical indicator.

17. The system of claim 16 wherein said interactive video game is configured to simulate the receiving of the input signal following engagement of an anesthesia mode, even when said sensor module is no longer detecting control commands from the patient.

18. The system of claim 17 wherein the simulation is based on the previously received control commands from the patient.

19. A method of preparing a user during the administration of an inhalant, the method comprising:
disposing over a predetermined area of the user's face a mask;
attaching an adapter housing to the mask,
activating a sensor module disposed within the adapter housing,
communicatively linking the sensor module with a computing device,
receiving, by the sensor module, control commands generated by the user,
transmitting the control commands from the sensor module to the computing device, and
presenting interactive media to the user, the interactive media being displayed on the computing device and configured to respond to the control commands generated by the user.

20. The method of claim 19 wherein said attaching step comprises attaching the adapter housing to an external surface of the mask.
